(19) United States
(12) Patent Application Publication
(10) Pub. No.: US 2025/0381111 A1
(43) Pub. Date: Dec. 18, 2025

(54) COSMETIC COMPOSITIONS CONTAING LAURYL LAURATE AND SEMICRYSTALLINE POLYMERS

(71) Applicant: L'OREAL, Paris (FR)

(72) Inventors: Nicholas David STEBBINS, Clark, NJ (US); Jonathan James FAIG, Sayreville, NJ (US)

(73) Assignee: L'OREAL, Paris (FR)

(21) Appl. No.: 18/746,183

(22) Filed: Jun. 18, 2024

(51) Int. Cl. A61K 8/37; A61K 8/81; A61Q 19/00

(52) U.S. Cl. CPC A61K 8/37; A61K 8/8152; A61Q 19/00; A61K 2800/48; A61K 2800/54; A61K 2800/592

(57) ABSTRACT

The instant disclosure relates to a composition, especially cosmetic composition, in particular for caring for keratin materials, comprising: lauryl laurate and at least one semicrystalline polymer containing at least one alkyl acrylate chain.

### FIELD OF THE DISCLOSURE

The instant disclosure relates to cosmetic compositions that include lauryl laurate and one semicrystalline polymer containing at least one alkyl acrylate chain and to methods for treating skin with the cosmetic composition.

### BACKGROUND

Many compositions, especially cosmetic compositions, have been developed for easy and comfortable application onto a targeted substrate. Unfortunately, many of these compositions are in fact difficult to apply and do not possess a smooth feel upon application. Moreover, compositions often have a tendency to feel tacky, yielding poor application and spreadability characteristics. The tacky and sticky feel is undesirable to consumers. Several approaches, such as using light emollients, powders, or combinations thereof may reduce tackiness; however, the resulting cosmetic compositions may not provide sufficient consumer appeal and may still have residual tackiness that can be felt on the skin after application. Therefore, it is desirable to provide a composition without having a tacky feel, easily spread and that is pleasing to consumers.

A cosmetic composition and methods of using and producing cosmetic compositions that do not suffer from one or more of the above drawbacks would be desirable in the art.

There is a need for compositions that are not tacky/sticky, not too thick, not too rich, not too draggy and not too greasy. The inventors of the instant disclosure discovered unique cosmetic compositions and method of using the compositions that address these and other needs.

### SUMMARY

The instant disclosure relates to cosmetic compositions that contain lauryl laurate and semicrystalline polymers containing at least one alkyl acrylate chain. The compositions are stable, have pleasant tactile properties, are aesthetically appealing, and easy to use. The unique combination provides a reduction in sticky feeling in an emulsion-based skin care formula without adding negative sensory attributes.

One way to mitigate tack is to add insoluble powders or fillers (e.g., silica, cellulose), but the risk of drag and piling upon application increases. Additionally, heavier oils and higher levels of oils can also help cover tack, at the expense of a formula feeling too greasy and appearing too shiny. While certain waxy ingredients (e.g., fatty alcohols/acids, high melting natural waxes) can cover tack, they also can leave too much of a waxy, draggy residue.

The inventors discovered that by combining lauryl laurate and semicrystalline polymers containing at least one alkyl acrylate chain surprisingly eliminates or greatly reduces the tackiness caused by humectants, polymers, certain actives, polyols and the like.

The cosmetic composition include:
(a) lauryl laurate; and
(b) at least one semicrystalline polymer containing at least one alkyl acrylate chain.

In some cases, the composition is in the form of an oil-in-water emulsion or an water-inoil-emulsion.

In one or more embodiments, lauryl laurate is present from about 0.1 to about 5 wt. %, based on a total weight of the composition.

In some embodiments, the compositions may include at least one semicrystalline polymer containing at least one alkyl acrylate chain are chosen from the homopolymers obtained by polymerization of a monomer chosen from alkyl acrylates and alkyl methacrylates and from the copolymers obtained by copolymerization of a monomer chosen from alkyl acrylates and alkyl methacrylates with a hydrophilic monomer, preferably a hydroxyethyl (meth)acrylate, and mixtures thereof. In one or more embodiments, the semicrystalline polymer(s) containing at least one alkyl acrylate chain are chosen from a polystearyl acrylate, a polybehenyl acrylate, a behenyl acrylate/2-hydroxyethyl acrylate copolymer, a stearyl acrylate/2-hydroxyethyl acrylate copolymer, and one of the mixtures thereof. In some embodiments, the semicrystalline polymer(s) containing at least one alkyl acrylate chain at least one of the one or more intelimer is selected from the group consisting of Poly C10-30 Alkyl Acrylate, C12-22 Alkyl

Acrylate/Hydroxyethylacrylate Copolymer, and mixtures thereof. In one or more embodiments, the content of semicrystalline polymer(s) containing at least one alkyl acrylate chain is present from about 0.1 to about 3 wt. % based on a total weight of the composition.

In one or more embodiments, the compositions may further comprise one or more polymeric thickener. In various embodiments, the one or more polymeric thickeners are present from about 0.05 to about 5 wt. % based on a total weight of the composition.

In one or more embodiments, the compositions may further comprise one or more fatty compounds. In some embodiments, the one or more fatty compounds are present from about 0.1 to about 30 wt. % based on a total weight of the composition.

In various embodiments, the compositions may further comprise one or more surfactants. In some embodiments, the one or more surfactants are present from about 0.05 to about 15 wt. % based on a total weight of the composition.

In one or more embodiments, the compositions may further comprise one or more skin active agents. In some embodiments, the total amount of the skin active agents is from at least about 1 wt. % based on a total weight of the composition.

In one or more embodiments, the compositions may further comprise one or more water soluble solvents. In some embodiments, one or more water soluble solvents are selected form the group consisting of glycerin, C2-C5 mono-alcohols, polyols (polyhydric alcohols), glycols, and a mixture thereof. In one or more embodiments, the one or more water soluble solvents are present from about 0.1 to about 30 wt. % based on a total weight of the composition.

Another aspect of the instant disclosure can include a cosmetic composition comprising:
(a) about 0.1 to about 5 wt. % of lauryl laurate;
(b) about 0.1 to about 3 wt. % poly C10-30 alkyl acrylate;
(c) about 0.05 to about 5 wt. % of one or more polymeric thickeners;
(d) about 0.05 to about 15 wt. % of one or more surfactants;
(e) about 0.05 to about 15 wt. % of one or more fatty compounds;
(f) about 0.1 to about 20 wt. % of one or more skin active agents;
(g) about 0.1 to about 30 wt. % of one or more water soluble solvents selected from glycerin, C2-C5 mono-alcohols, polyols (polyhydric alcohols), glycols, and a mixture thereof; wherein all weight percentages are based on a total weight of the composition.

Another aspect of the instant disclosure relates to method for treating skin. The methods include applying the cosmetic composition according to the instant disclosure to the skin, in particular the skin of the face.

It is to be understood that both the foregoing general description and the following detailed description are exemplary and explanatory only, and are not restrictive of the invention.

### DETAILED DESCRIPTION

The compositions of the instant case are particularly useful for treating skin, especially the skin of the face. The composition may be in the form of an oil-in-water emulsion or an oil-in-water emulsion. The compositions typically include:
(a) lauryl laurate;
(b) at least one semicrystalline polymer containing at least one alkyl acrylate chain.

### Lauryl Laurate

In some embodiments, the compositions may include lauryl laurate. For examples the compositions may include an amount of lauryl laurate ranging from about 0.1 wt. % to about 5 wt. %, about 0.15 wt. % to about 4.9 wt. %, about 0.2 wt. % to about 4.8 wt. %, about 0.25 wt. % to about 4.7 wt. %, about 0.3 wt. % to about 4.6 wt. %, about 0.35 wt. % to about 4.5 wt. %, about 0.4 wt. % to about 4.4 wt. %, about 0.45 wt. % to about 4.3 wt. %, about 0.5 wt. % to about 4.2 wt. %, about 0.55 wt. % to about 4.1 wt. %, about 0.6 wt. % to about 4.0 wt. %, about 0.7 wt. % to about 3.8 wt. %, about 0.8 wt. % to about 3.6 wt. %, about 1 wt. % to about 3.5 wt. %, about 1.5 wt. % to about 3 wt. %. including ranges and sub-ranges therebetween, based on the total weight of the composition.

### Semicrystalline Polymer Containing at Least One Alkyl Acrylate Chain

In various embodiments, the compositions may include at least one semicrystalline polymer containing at least one alkyl acrylate chain.

As indicated above, the composition according to the invention comprises at least one semi-crystalline polymer containing at least one alkyl acrylate chain, the alkyl radicals preferably being C16-C22.

For the purposes of the instant disclosure, the term “semi-crystalline polymer” is understood to mean polymers comprising a crystallisable portion, pendent chain or block in the backbone, and an amorphous portion in the backbone, and having a first-order reversible change of phase temperature, in particular melting point (solid-liquid transition).

When the crystallisable portion is a block of the polymer backbone, this crystallisable block is of different chemical nature from that of the amorphous blocks; the semi-crystalline polymer is, in this case, a block polymer, for example of the diblock, triblock or multiblock type, comprising at least one crystallisable block and at least one amorphous block.

The term “block” is generally understood to mean at least 5 identical repeating units. The crystallisable block(s) is (are) then of different chemical nature from the 5 amorphous block(s).

Advantageously, the semi-crystalline polymer containing at least one alkyl acrylate chain of the composition of the instant disclosure has an average molecular mass (Mn) of greater than or equal to about 2000 g/mol, preferably from about 2000 to 800 000 g/mol, preferably from about 3000 to 500 000 g/mol, preferably from about 4000 to 150 000 g/mol, and preferably from about 4000 to 99 000 g/mol.

Aside from the crystallisable chains or blocks, the blocks of the polymers are amorphous.

For the purposes of the instant disclosure, the term “crystallisable chain or block” is understood to mean a chain or block which, if it were alone, would change from the amorphous state to the crystalline state reversibly, depending on whether the temperature is above or below the melting point. For the purposes of the instant disclosure, a chain is a group of atoms, which is pendent or lateral relative to the polymer backbone. A block is a group of atoms belonging to the backbone, this group constituting one of the repeating units of the polymer. Advantageously, the “pendent crystallisable chain” may be a chain containing at least 6 carbon atoms.

Preferably, the polymer backbone of the semi-crystalline polymers is soluble in the oily phase.

Thus, the semi-crystalline polymer containing at least one alkyl acrylate chain that is used in the composition of the instant disclosure is generally introduced into the oily phase of the composition.

Preferably, the crystallisable blocks or chains of the semi-crystalline polymers containing at least one alkyl acrylate chain represent at least approximately 30% by weight and preferably at least approximately 40% by weight relative to the total weight of each polymer.

The semi-crystalline polymers comprising crystallisable blocks used according to the instant disclosure are block or multiblock polymers. They may be obtained by polymerising monomers bearing reactive (or ethylenic) double bonds or by polycondensation. When the polymers of the instant disclosure are polymers bearing crystallisable side chains, these polymers are advantageously in random or statistical form.

The semi-crystalline polymers of the instant disclosure may be of synthetic origin. Moreover, they do not comprise a polysaccharide backbone.

The semi-crystalline polymers of the composition of the instant disclosure may or may not be partially crosslinked. It may then be a case of chemical crosslinking, by reaction with a multifunctional monomer during the polymerisation. It may also be a case of physical crosslinking, which may then be due either to the establishment of bonds of hydrogen or dipolar type between groups borne by the polymer, such as dipolar interactions between carboxylate ionomers, these interactions being in small amount and borne by the polymer backbone; or due to a phase separation between the crystallisable blocks and the amorphous blocks borne by the polymer.

Preferably, the semi-crystalline polymers containing at least one alkyl acrylate chain of the composition according to the instant disclosure are non-crosslinked.

As examples of semi-crystalline polymers according to the instant disclosure containing at least one alkyl acrylate chain, mention may be made of those resulting from the polymerisation of one or more saturated alkyl (meth)acrylates.

Preferably, the semi-crystalline polymers bearing a crystallisable side chain are alkyl (meth)acrylate homopolymers, or copolymers of these monomers with a hydrophilic monomer.

According to a particular embodiment of the instant disclosure, the semi-crystalline polymer containing at least one alkyl acrylate chain is derived from a monomer bearing a crystallisable chain chosen from saturated alkyl (meth)acrylates and even more particularly poly(stearyl acrylate) s or poly(behenyl acrylate) s.

According to another particular embodiment of the instant disclosure, the semi-crystalline polymer containing at least one alkyl acrylate chain is chosen from copolymers resulting from the polymerisation of at least one monomer bearing a crystallisable chain and chosen from saturated alkyl (meth)acrylates.

According to a preferred embodiment, the semi-crystalline polymer containing at least one alkyl acrylate chain is chosen from the homopolymers obtained by polymerisation of a monomer chosen from alkyl acrylates and alkyl methacrylates and from the copolymers obtained by copolymerisation of a monomer chosen from alkyl acrylates and alkyl methacrylates with a hydrophilic monomer, preferably N-vinylpyrrolidone or a hydroxyethyl (meth)acrylate, and mixtures thereof, more preferentially still a hydroxyethyl (meth)acrylate, and mixtures thereof.

As particular examples of semi-crystalline polymers that can be used in the composition according to the instant disclosure, mention may be made of the homopolymers obtained by polymerisation of a monomer chosen from alkyl acrylates and alkyl methacrylates, preferably from stearyl acrylates and behenyl acrylates, especially the polymers having the INCI name Poly C10-30 Alkyl Acrylate such as the Intelimer® products from Evonik, such as the product Intelimer® IPA 13-1, which is a polystearyl acrylate, or the product Intelimer® IPA 13-6, which is a behenyl polymer (polybehenyl acrylate), or else the Tego® SP products from Evonik.

As particular examples of semi-crystalline polymers that can be used in the composition, mention may also be made of the copolymers obtained by copolymerisation of a monomer chosen from alkyl acrylates and alkyl methacrylates with a hydrophilic monomer, preferably a hydroxyethyl (meth)acrylate, especially the copolymers described in applications FR 3 073 405 and FR 3 073 406, and preferably the copolymers obtained by copolymerisation of at least one monomer chosen from alkyl acrylates, preferably from behenyl acrylates, stearyl acrylates, and mixtures thereof, with a hydrophilic monomer such as hydroxyethyl (meth)acrylate and N-vinylpyrrolidone, and preferably hydroxyethyl acrylate.

Preferably, the semi-crystalline polymer containing at least one alkyl acrylate chain is a behenyl acrylate/2-hydroxyethyl acrylate copolymer, a stearyl acrylate/2-hydroxyethyl acrylate copolymer and/or a mixture thereof, and in particular the polymer with the INCI name: Poly C12-22 Alkyl Acrylate/Hydroxyethyl Acrylate Copolymer sold in particular under the name Tego SP Senstar by Evonik.

According to a preferred embodiment, the semi-crystalline polymer(s) containing at least one alkyl acrylate chain are chosen from a polystearyl acrylate, a polybehenyl acrylate, a behenyl acrylate/2-hydroxyethyl acrylate copolymer, a stearyl acrylate/2-hydroxyethyl acrylate copolymer, and a mixture thereof, in particular chosen from the polymer with the INCI name: Poly C10-30 Alkyl Acrylate, the copolymer with the INCI name C12-22 Alkyl Acrylate/Hydroxyethyl Acrylate Copolymer, and mixtures thereof, more particularly the polymer with the INCI name: C12-22 Alkyl Acrylate/Hydroxyethyl Acrylate Copolymer.

According to a more preferred embodiment, the semi-crystalline polymer containing at least one alkyl acrylate chain is a polymer having the INCI name: C12-22 Alkyl Acrylate/Hydroxyethyl Acrylate Copolymer.

More particularly, the composition in accordance with the invention comprises, as semi-crystalline polymer, at least one lipophilic acrylic polymer containing monomer units of formulae (A) and (B):

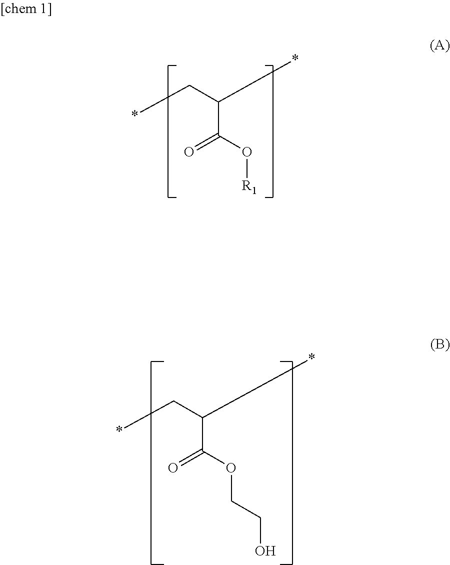

in which:
R₁, independently at each instance, is chosen from alkyl or alkyenyl radicals, and
at least 60% by weight of the R₁ groups are radicals chosen from stearyl and behenyl radicals, the percentage by weight relating to the sum of all the R₁ groups present in the polymer,
and
the weight ratio of the sum of all the hydroxyethyl acrylate units to the sum of all the acrylate units bearing the R₁ group ranges from 1:30 to 1:1,
and the sum of the total of units A and B is at least 95% by weight of the total weight of the polymer.

Preferably, R₁ is constituted of alkyl radicals, preferably of C₁₆-C₂₂ alkyl radicals, and more preferentially of stearyl (C₁₈) radicals or of behenyl (C₂₂) radicals.

Preferably, at least 70% by weight of the R₁ groups are stearyl or behenyl radicals, preferentially at least 80% by weight and more preferentially at least 90% by weight.

According to one preferred embodiment, all the R₁ groups are behenyl radicals.

According to another preferred embodiment, all the R₁ groups are stearyl radicals.

Preferably, the weight ratio of the sum of all the hydroxyethyl acrylate units to the sum of all the acrylate units bearing the R₁ group ranges from 1:15 to 1:1, preferably ranges from 1:10 to 1:4.

The polymer has a number-average molecular weight Mn ranging from 2000 to 9000 g/mol, preferably ranging from 5000 to 9000 g/mol. The number-average molecular weight can be measured by the gel permeation chromatography method, for example according to the method described in the example hereinbelow.

Preferably, the polymer has a melting point ranging from 40° C. to 70° C. and preferentially ranging from 45° C. to 67° C. The melting point is measured by differential scanning calorimetry (DSC), for example according to the method described in the example hereinbelow.

According to a first embodiment, when the polymer is such that at least 60% by weight of the R₁ groups are stearyl radicals, then the polymer preferably has a melting point ranging from 40 to 60° C., and preferentially ranging from 45 to 55° C.

According to a second embodiment, when the polymer is such that at least 60% by weight of the R₁ groups are behenyl radicals, then the polymer has a melting point ranging from 60° C. to 70° C., and preferentially ranging from 63° C. to 67° C.

The polymer used according to the invention can be prepared by polymerisation of a monomer having the formula below:

CH₂═CH—COO—R₁,

The polymerisation may be performed according to known methods, such as solution polymerisation or emulsion polymerisation.

The polymerisation is, for example, described in document US 2007/0264204.

The total amount of the at least one semi-crystalline polymers will vary.

The semi-crystalline polymer(s) containing at least one alkyl acrylate chain may be present in the composition according to the instant disclosure in an active material content ranging from about 0.1 wt. % to about 3 wt. % based on a total weight of the composition.

In some embodiments, the at least one or more semi-crystalline polymers is present from about 0.1 to about 2.9 wt. %, about 0.1 wt. % to about 2.8 wt. %, about 0.1 wt. % to about 2.6 wt. %, about 0.1 wt. % to about 2.4 wt. %, about 0.1 wt. % to about 2.2 wt. %, about 0.1 wt. % to about 2.0 wt. %, about 0.1 wt. % to about 1.8 wt. %, about 0.2 wt. % to about 3 wt. %, about 0.2 wt. % to about 2.9 wt. %, about 0.2 wt. % to about 2.8 wt. %, about 0.2 wt. % to about 2.6 wt. %, about 0.2 to about 2.4 wt. %, about 0.2 wt. % to about 2.2 wt. %, about 0.2 wt. % to about 2.0 wt. %, about 0.2 wt. % to about 1.8 wt. %, about 0.3 wt. % to about 3 wt. %, about 0.3 wt. % to about 2.9 wt. %, about 0.3 wt. % to about 2.8 wt. %, about 0.3 wt. % to about 2.6 wt. %, about 0.3 to about 2.4 wt. %, about 0.3 wt. % to about 2.2 wt. %, about 0.3 wt. % to about 2.0 wt. %, about 0.3 wt. % to about 1.8 wt. %, about 0.4 wt. % to about 3.0 wt. %, 0.4 to about 2.9 wt. %, about 0.4 wt. % to about 2.8 wt. %, about 0.4 wt. % to about 2.6 wt. %, about 0.4 wt. % to about 2.4 wt. %, about 0.4 wt. % to about 2.2 wt. %, about 0.4 wt. % to about 2.0 wt. %, about 0.4 wt. % to about 1.8 wt. %, about 0.5 wt. % to about 3.0 wt. %, 0.5 to about 2.9 wt. %, about 0.5 wt. % to about 2.8 wt. %, about 0.5 wt. % to about 2.6 wt. %, about 0.5 wt. % to about 2.4 wt. %, about 0.5 wt. % to about 2.2 wt. %, about 0.5 wt. % to about 2.0 wt. %, or about 0.5 wt. % to about 1.8 wt. %, including all ranges and sub-ranges there between, based on the total weight of the composition.

Accordingly, the amount of the at least one or more semi-crystalline polymers in the composition according to the instant disclosure may be from about 0.1, 0.15, 0.2, 0.25, 0.3, 0.35, 0.4, 0.5, 0.6, 0.7, 0.8, 0.9, 1.0, 1.2, 1.4, 1.6, 1.8, 2.0, 2. 2 to about 2.2, 2.4, 2.6, 2.8, 3.0 wt. %, including all ranges and sub-ranges there between, based on the total weight of the composition.

### Polymeric Thickeners

In various embodiments, the compositions may include one or more polymeric thickeners.

According to the instant disclosure, the suitable polymeric thickeners of the instant disclosure could be selected from acrylic-or acrylate based polymers.

The polymers are pre-neutralized and preferably selected from taurate polymers. Such polymers comprise an ionic monomer portion, 2-acrylamido-2-methylpropane sulfonic acid (AMPS), as well as a further, less polar monomer portion (vinylpyrrolidone or beheneth-25 methacrylate). These polymers are used as polymeric thickeners.

Examples of taurate polymers are Acrylates/Vinyl Isodecanoate Crosspolymer (Stabylen 30 from 3V), Ammonium Acryloyldimethyltaurate/VP Copolymer (Aristoflex AVC from Clariant), Ammonium Acryloyldimethyltaurate/Beheneth-25 Methacrylate Crosspolymer (Aristoflex HMB from Clariant), Acrylates/Ceteth-20 Itaconate Copolymer (Structure 3001 from National Starch), Polyacrylamide (Sepigel 305 from SEPPIC), Non-ionic thickener, (Aculyn 46 from Rohm and Haas), or mixtures thereof.

Anionic polymers may be polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself.

The anionic polymers may comprise at least one hydrophilic unit of olefinic unsaturated carboxylic acid type, and at least one hydrophobic unit exclusively of (C10-C30)alkyl ester of unsaturated carboxylic acid type.

Anionic polymers useful herein include, for example: Polyacrylic acid; Polymethacrylic acid; Carboxyvinylpolymer; acrylate copolymers such as Acrylate/C10-30 alkyl acrylate crosspolymer, Acrylic acid/vinyl ester copolymer/AcrylatesNinyl Isodecanoate crosspolymer, Acrylates/Palmeth-25 Acrylate copolymer, Acrylate/Steareth-20 Itaconate copolymer, and Acrylate/Celeth-20 Itaconate copolymer;

sulfonate polymers such as Polysulfonic acid, Sodium Polystyrene Sulfonate supplied from Akzo Nobel under the tradename FLEXAN II, copolymers of methacrylic acid and acrylamidomethylpropane sulfonic acid, and copolymers of acrylic acid and acrylamidomethylpropane sulfonic acid; carboxymethycellulose; carboxy guar gum; copolymers of ethylene and maleic acid; and acrylate silicone polymer. In some instances, the anionic polymers include, for example, Carbomer supplied from Noveon under the tradename CARBOPOL 981 and CARBOPOL 980; Acrylates/C10-30 Alkyl Acrylate Crosspolymer having tradenames Pemulen TR-1, PEMULEN TR-2, CARBOPOL 1342, CARBOPOL 1382, and CARBOPOL ETD 2020, all available from Noveon; sodium carboxymethylcellulose supplied from Hercules as CMC series; and Acrylate copolymer having a tradename Capigel supplied from Seppic; Carbomers, acrylates copolymer having the tradename CARBOPOL Aqua SF-1 and available from Lubrizol as an aqueous dispersion, and acrylates crosspolymer-4 having the tradename CARBOPOL Aqua SF-2 and available from Lubrizol as an aqueous dispersion.

In an embodiment, the anionic polymer of the instant disclosure is carbomer which may be commercially available from the supplier Lubrizol under the tradename of CARBOPOL 980. Exemplary of non-ionic polymers could be as follows:
(i) hydroxyethylcellulose, for instance the product NATROSOL 250 HHR PC or NATROSOL 250 HHR CS sold by the company Ashland;
(ii) celluloses modified with groups comprising at least one fatty chain; examples that may be mentioned include:
   hydroxyethylcelluloses modified with groups comprising at least one fatty chain, such as alkyl, arylalkyl or alkylaryl groups, or mixtures thereof, and in which the alkyl groups are preferably C8-C22, for instance the product NATROSOL Plus Grade 330 CS (C16 alkyls) sold by the company Ashland, or the product BERMOCOLL EHM 100 sold by the company AkzoNobel; methyl hydroxyethylcellulose; methyl ethyl hydroxyethylcellulose, known as the product STRUCTURE CEL 8000 M sold by the company AkzoNobel; or hydroxypropyl cellulose, known as the product KLUCEL MF PHARM HYDROXYPROPYLCELLULOSE sold by the company Ashland;
   hydroxyethylcelluloses modified with alkylphenyl polyalkylene glycol ether groups, such as the product Amercell Polymer HM-1500 (nonylphenyl polyethylene glycol (15) ether) sold by the company Amerchol; or
(iii) hydroxypropyl guars such as hydroxypropyl guar sold by as the product JAGUAR HP 105 by the company Rhodia and hydroxypropyl guars modified with groups comprising at least one fatty chain, such as the product Esaflor HM 22 (C22 alkyl chain) sold by the company Lamberti, and the products RE210-18 (C14 alkyl chain) and RE205-1 (C20 alkyl chain) sold by the company Rhodia

In some embodiments, the polymeric thickener may be a hydrophilic acrylic polymer. In some embodiments, the polymeric thickener is selected from the group consisting of Acrylates/C10-30 Alkyl Acrylates, Ammonium Polyacryloyldimethyl Taurate, Ammonium Acryloyldimthyltaurate/Vp Copolymer, Sodium Acryloyldimthyltaurate/Vp Copolymer and combinations thereof.

The total amount of the one or more polymeric thickeners will vary. Nonetheless, in various embodiments, the total amount of the one or more polymeric thickeners is about 0.05 to about 5 wt. %, based on the total weight of the composition. In further embodiments, the total amount of the one or more polymeric thickeners is from about 0.05 to about 4 wt. %, about 0.05 wt. % to about 3 wt. %, about 0.05 wt. % to about 2 wt. %, about 0.1 to about 5 wt. %, about 0.1 wt. % to about 4.5 wt. %, about 0.1 wt. % to about 4 wt. %, about 0.1 wt. % to about 3.5 wt. %, about 0.1 wt. % to about 3 wt. %, about 0.1 wt. % to about 2.5 wt. % or about 0.1 wt. % to about 2 wt. %, including all ranges and sub-ranges there between, based on the total weight of the composition.

Accordingly, the amount of one or more polymeric thickeners in the composition according to the instant disclosure may be from about 0.05, 0.1, 0.15, 0.2, 0.25, 0.3, 0.35, 0.4, 0.5, 0.6, 0.7, 0.8, 0.9, 1.0, 1.2, 1.4, 1.6, 1.8, 2.0, 2. 2 to about 2.2, 2.4, 2.6, 2.8, 3.0, 3.1, 3.2, 3.4, 3.6, 3.8, 4.0, 4.2, 4.4, 4.6, 4.8 or 5.0 wt. %, including all ranges and sub-ranges there between, based on the total weight of the composition.

### Fatty Compounds

In various embodiments, the compositions may include one or more fatty compounds.

Fatty compounds include, for example, oils. Non-limiting examples of oils include silicone oils, fluoro oils, hydrocarbon-based oils, etc. In some instances, the cosmetic composition includes oils that are plant based or vegetable-based oils. The amount of oil and/or plant based or vegetable based oil present in the cosmetic composition may be from about 0.1 to about 30 wt. %, about 0.1 to about 25 wt. %, about 0.1 to about 20 wt. %, about 0.1 to about 15 wt. %, about 0.1 to about 10 wt. %, about 0.5 to about 30 wt. %, about 0.5 to about 25 wt. %, about 0.5 to about 20 wt. %, about 0.5 to about 15 wt. %, about 0.5 to about 10 wt. %, about 1 wt. % to about 30 wt. %, about 1 wt. % to about 25 wt. %, about 1 wt. % to about 20 wt. %, about 1 wt. % to about 15 wt. %, about 1 wt. % to about 10 wt. %, about 1.5 wt. % to about 30 wt. %, about 1.5 wt. % to about 25 wt. %, about 1.5 wt. % to about 20 wt. %, about 1.5 wt. % to about 15 wt. %, about 1.5 wt. % to about 10 wt. %, about 2 wt. % to about 30 wt. %, about 2 wt. % to about 25 wt. %, about 2 wt. % to about 20 wt. %, about 2 wt. % to about 15 wt. %, about 2 wt. % to about 10 wt. %, about 3 wt. % to about 30 wt. %, about 3 wt. % to about 25 wt. %, about 3 wt. % to about 20 wt. %, about 3 wt. % to about 15 wt. %, about 3 wt. % to about 10 wt. %, about 4 wt. % to about 30 wt. %, about 4 wt. % to about 25 wt. %, about 4 wt. % to about 20 wt. %, about 4 wt. % to about 15 wt. %, about 4 wt. % to about 10 wt. %, about 5 wt. % to about 30 wt. %, about 5 wt. % to about 25 wt. %, about 5 wt. % to about 20 wt. %, about 5 wt. % to about 15 wt. %, about 5 wt. % to about 10 wt. %, about 6 wt. % to about 30 wt. %, about 6 wt. % to about 25 wt. %, about 6 wt. % to about 20 wt. %, about 6 wt. % to about 15 wt. %, or about 6 wt. % to about 10 wt. %, including all ranges and sub-ranges there between, based on the total weight of the composition.

The term “oil” means any fatty substance which is in liquid form at room temperature (20-25° C.) and at atmospheric pressure (760 mmHg). Often, at least one of the oils in the cosmetic composition is part of an oily phase. An “oily phase” is a phase comprising at least one oil that may include additional liposoluble and lipophilic ingredients and the fatty substances. Oil that is suitable for use herein may be volatile or non-volatile. The term “volatile oil” relates to an oil that is capable of evaporating on contact with the skin or a keratin fiber in less than one hour, at room temperature and atmospheric pressure. Volatile oil(s) are liquid at room temperature and have a non-zero vapor pressure, at room temperature and atmospheric pressure, ranging in particular from 0.13 Pa to 40 000 Pa (10⁻³ to 300 mmHg). The term “non-volatile oil” relates to an oil that remains on the skin or the keratin fiber, at room temperature and atmospheric pressure, for at least several hours and which in particular has a vapor pressure of less than 10⁻³ mmHg (0.13 Pa).

The term “silicone oil” relates to oil comprising at least one silicon atom, and especially at least one Si-O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil or a synthetic hydrocarbon-based oil. Further, suitable oil may be a mineral hydrocarbon-based oil, a plant hydrocarbon-based oil, or a synthetic hydrocarbon-based oil.

### Silicone Oils

The cosmetic compositions described herein may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes

(PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl (trimethylsiloxy) diphenylsiloxanes, diphenyl dimethicones, diphenyl (methyldiphenyl) trisiloxanes or (2-phenylethyl) trimethylsiloxysilicates.

Other examples of silicone oils that may be mentioned include volatile linear or cyclic silicone oils, especially those with a viscosity 8 centistokes (8x106 m2/s) and especially containing from 2 to 7 silicon atoms, these silicones optionally comprising alkyl or alkoxy groups containing from 1 to 10 carbon atoms. As volatile silicone oils that may be used in the instant disclosure, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

### Fluoro Oils

The cosmetic compositions described herein may comprise one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

### Hydrocarbon-Based Oils

The cosmetic compositions described herein may comprise one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the compositions include a synthetic oil selected from the group consisting of hydrogenated polyisobutene and hydrogenated polydecene.

The hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 4 0 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ≥10, for instance Purcellin oil (cetearyl octanoate), isopropyl myristate, isopropyl palmitate, C₁₂-C₁₅ alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C₁₂-C₁₃ alkyl) tartrates, such as those sold under the name Cosmacol ETI® by Enichem Augusta Industriale, and also di(linear C₁₄-C₁₅ alkyl) tartrates, such as those sold under the name Cosmacol ETL® by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid or linolenic acid;
(vii) carbonates, such as dicaprylyl carbonate, such as the product sold under the name Cetiol CC@ by Cognis;
(viii) fatty amides, such as isopropyl N-lauroyl sarcosinate, such as the product sold under the trade name Eldew SL 205® from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

In certain instances, the non-volatile hydrocarbon-based oils are glyceride triesters and in particular to caprylic/capric acid triglycerides, synthetic esters and in particular isononyl isononanoate, oleyl erucate, C₁₂-C₁₅ alkyl benzoate, 2-ethylphenyl benzoate and fatty alcohols, such as octyldodecanol.

As volatile hydrocarbon-based oils, mention is made of hydrocarbon-based oils containing from 8 to 16 carbon atoms and in particular of branched C₈-C₁₆ alkanes, such as C₈-C₁₆ isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C₈-C₁₆ esters, and isohexyl neopentanoate.

Preferably, the oil is chosen from plant based or vegetable based oils, such as coconut oil, soybean oil, canola oil, rapeseed oil, corn oil, cottonseed oil, olive oil, palm oil, peanut oil, safflower oil, sesame oil, sunflower oil, linseed oil, palm kernel oil, tung oil, jatropha oil, mustard oil, camelina oil, pennycress oil, castor oil, wheatgerm oil, apricot kernel oil, pistachio oil, poppy oil, pine oil, avocado oil, hazel nut oil, grapeseed oil, colza oil, cade oil, peach kernel oil, coffee bean oil, jojoba oil, and a mixture thereof.

Non-limiting examples of fatty compounds include:
i. hydrocarbon oils of animal origin such as perhydrosqualene;
ii. plant hydrocarbon oils, such as liquid triglycerides of fatty acids, for example sunflower oil, corn oil, soybean oil, marrow oil, grapeseed oil, sesame oil, hazelnut oil, apricot oil, macadamia oil, castor oil, avocado oil, caprylic/capric acid triglycerides;
iii. oils of formula R₉COOR₁₀ in which R₉ represents a higher fatty acid residue containing from 7 to 19 carbon atoms and R₁₀ represents a branched hydrocarbon chain containing from 3 to 20 carbon atoms, such as, for example, Purcellin oil;
iv. linear or branched hydrocarbons of mineral or synthetic origin, such as non-volatile liquid paraffins and derivatives thereof, petroleum jelly (petrolatum), polydecenes, and hydrogenated polyisobutene such as parleam;
v. synthetic esters and ethers such as isopropyl myristate, octanoates, decanoates or ricinoleates of alcohols or of polyalcohols;
vi. fatty alcohols such as octyidodecanol or oleyl alcohol;
vii. partially hydrocarbonated and/or siliconated fluoro oils;
viii. silicone oils such as linear, non-volatile polydimethylsiloxanes (dimethicone) which are liquid or pasty at room temperature, phenyldimethicones, phenyltrimethicones and polymethylphenylsiloxanes; and mixtures thereof.

In some instances, the one or more fatty compounds may be selected form polyolefins (petrolatum), waxes, squalane, squalene, hydrogenated polyisobutene, hydrogenated polydecene, polybutene, mineral oil, pentahydrosqualene, dimethicone, and a mixture thereof.

Additional fatty compounds that are worth mentioning include fatty alcohols, fatty esters, fatty alcohols derivatives, fatty acid derivatives, such as those discussed below.

### Fatty Alcohols

The one or more fatty compounds may be glycerolated and/or oxyalkylenated, include from 8 to 30 carbon atoms, and/or be saturated or unsaturated. The fatty alcohols useful herein include those having from about 8 to about 30 carbon atoms, from about 12 to about 22 carbon atoms, and from about 14 to about 22 carbon atoms.

These fatty alcohols can be straight or branched chain alcohols and can be saturated or unsaturated. Non-limiting examples of fatty alcohols include decyl alcohol, undecyl alcohol, dodecyl alcohol, myristyl alcohol, cetyl alcohol, stearyl alcohol, isostearyl alcohol, isocetyl alcohol, behenyl alcohol, linalool, oleyl alcohol, cis4-t-butylcyclohexanol, myricyl alcohol and a mixture thereof. In some cases, the fatty alcohols comprise at least one of or may be chosen from myristyl alcohol, cetyl alcohol, stearyl alcohol, isostearyl alcohol, oleyl alcohol, isotridecyl alcohol, and a mixture thereof.

The saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

The unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

Non-limiting examples of solid fatty alcohols include linear or branched, saturated or unsaturated alcohols containing from 8 to 30 carbon atoms, for example, myristyl alcohol, cetyl alcohol, stearyl alcohol and their mixture, cetylstearyl alcohol.

### Fatty Esters

The fatty compounds of the skin tightening composition may be liquid or solid fatty esters at 25° C., 1 atm. The fatty esters may include esters from a C₆-C₃₂ fatty acid and/or a C₆-C₃₂ fatty alcohol. For example, the fatty compounds may include or be chosen from fatty acid monoesters and diesters, polyol esters, polyglycerol esters, polyglycerol polyricinoleate, polyglycerol poly-12-hydroxystearate, polyglycerol dimerate isostearate, ethylhexanoate, polyglycerol esters, and a mixture thereof. These esters may be esters of saturated or unsaturated, linear or branched C₁-C₂₆ aliphatic mono or polyacids and of saturated or unsaturated, linear or branched C₁-C₂₅ aliphatic mono or polyalcohols, the total number of carbon atoms in the esters being greater than or equal to 10. In one instance, the fatty compounds comprise one or more fatty acid monoesters. For the esters of monoalcohols, at least one of the alcohol or the acid from which the esters result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate and isostearyl neopentanoate.

In some instances, the fatty esters are cetyl esters, such as esters of saturated fatty acids and fatty alcohols. For example, the fatty esters may include or be chosen from cetyl palmitate, cetyl stearate, myristyl myristate, myristyl stearate, cetyl myristate, stearyl stearate, cetearyl ethylhexanoate, and mixtures thereof. In one instance, the fatty esters may be one or more of or chosen from isopropyl isostearate, n-propyl myristate, isopropyl myristate, hexyl laurate, hexadecyl isostearate, hexydecyl laurate, hexyldecyl octanoate, n-propyl palmitate, isopropyl palmitate, and mixtures thereof. In another instance, the fatty esters include or may be chosen from diisobutyl adipate, 2-hexyldecyl adipate, di-2-heptylundecyl adipate, monoisostearic acid N-alkyl glycol, isocetyl isostearate, trimethylolpropane triisostearate, ethylene glycol di-2-ethylhexanoate, cetyl 2-ethylhexanoate, trimethylolpropane tri-2-ethylhexanoate, pentaerythritol tetra-2-ethylhexanoate, cetyl octanoate, octyldodecyl gum ester, oleyl oleate, octyldodecyl oleate, decyl oleate, neopentyl glycol dicaprate, triethyl citrate, 2-ethylhexyl succinate, isocetyl stearate, butyl stearate, diisopropyl sebacate, di-2-ethylhexyl sebacate, cetyl lactate, myristyl lactate, isopropyl palm itate, 2-ethylhexyl palm itate, 2-hexyldecyl palm itate, 2-heptylundecyl palm itate, cholesteryl 12-hydroxystearate, dipentaerythritol fatty acid ester, isopropyl myristate, octyldodecyl myristate, 2-hexyldecyl myristate, myristyl myristate, hexyldecyl dimethyloctanoate, ethyl laurate, hexyl laurate, diisostearyl malate, dicaprylyl carbonate, cetearyl ethylhexanoate, and mixtures thereof. In yet a further instance, the skin-tightening composition includes one or more of or may have fatty compounds chosen from cetearyl alcohol, cetearyl ethylhexanoate, isopropyl myristate, and mixtures thereof.

Non-limiting examples of solid fatty acid esters and/or fatty acid esters that may be mentioned include solid esters obtained from C₉-C₂₆ fatty acids and from C₉-C₂₅ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

Non-limiting examples of liquid fatty acid include triglyceride oils of plant or synthetic origin, such as liquid fatty acid triglycerides containing 6 to 30 carbon atoms, for instance heptanoic or octanoic acid triglycerides, or alternatively, e.g., sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camellina oil, tamanu oil, babassu oil and pracaxi oil, jojoba oil, shea butter oil, and mixtures thereof. In one instance, the one or more fatty compounds include at least one of or are selected from fatty acid triglycerides, oils, mineral oil, alkanes, fatty alcohols, fatty acids, fatty alcohol derivatives, alkoxylated fatty acids, polyethylene glycol esters of fatty acids, propylene glycol esters of fatty acids, butylene glycol esters of fatty acids, esters of neopentyl glycol and fatty acids, polyglycerol/glycerol esters of fatty acids, glycol diesters, diesters of ethylene glycol and fatty acids, esters of fatty acids and fatty alcohols, esters of short chain alcohols and fatty acids, esters of fatty alcohols, hydroxy-substituted fatty acids, waxes, and a mixture thereof. In another instance, fatty compounds of the skin-tightening composition includes one or more fatty acid triglycerides, such as caprylic/capric triglyceride.

### Fatty Alcohol Derivatives

The compositions may, in some instances, include fatty alcohol derivatives such as alkyl ethers of fatty alcohols, alkoxylated fatty alcohols, alkyl ethers of alkoxylated fatty alcohols, esters of fatty alcohols and a mixture thereof. Non-limiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof. Liquid fatty ethers may be chosen from liquid dialkyl ethers such as dicaprylyl ether. The non-liquid fatty ethers may also be chosen from dialkyl ethers and in particular dicetyl ether and distearyl ether, alone or as a mixture.

Esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate; diisopropyl sebacate; diisopropyl adipate; di-n-propyl adipate; triisopropyl citrate; glyceryl trilactate; glyceryl trioctanoate; neopentyl glycol diheptanoate; and diethylene glycol diisononanoate.

### Fatty Acid Derivatives

The compositions may, in some instances, include fatty acid derivatives. The fatty acid derivatives are defined herein to include fatty acid esters of the fatty alcohols as defined above, fatty acid esters of the fatty alcohol derivatives as discussed above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof.

### Surfactants

The various compositions described herein may include one or more surfactants, including cationic, anionic, non-ionic and/or amphoteric/zwitterionic surfactants. Non-limiting examples of surfactants that may be used are provided below.

The surfactants may be chosen from nonionic, anionic, cationic and amphoteric surfactants, and mixtures thereof. Reference may be made to the Kirk-Othmer Encyclopedia of Chemical Technology, volume 22, pages 333-432, 3rd Edition, 1979, Wiley, for the definition of the emulsifying properties and functions of surfactants, in particular pages 347-377 of this reference, for anionic, amphoteric and nonionic surfactants.

### Nonionic Surfactant

In one embodiment, the composition according to the instant disclosure comprises at least one nonionic surfactant.

The nonionic surfactants may in particular be chosen from alkyl and polyalkyl esters of poly(ethylene oxide), oxyalkylenated alcohols, alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, in particular sucrose stearate, glyceryl esters, optionally polyoxyethylenated alkyl and polyalkyl esters of glycerol, optionally polyoxyethylenated alkyl and polyalkyl ethers of glycerol, gemini surfactants, cetyl alcohol, stearyl alcohol, nonionic triblock poly(ethylene glycol)-block-poly(propylene glycol)-block-poly(ethylene glycol) copolymers, also referred to as poloxamers, and mixtures thereof.

As glyceryl esters, mention may notably be made of C₁₆-C₂₂ fatty acid esters of glycerol, in particular glyceryl esters of a fatty acid having 18 carbon atoms, and polyglyceryl esters of a fatty acid containing from 8 to 20 carbon atoms.

Glyceryl esters of a C₁₈ fatty acid that may notably be mentioned include glyceryl stearate (glyceryl monostearate, distearate and/or tristearate) (CTFA name: glyceryl stearate), glyceryl ricinoleate, or mixtures thereof.

As fatty acid ester of glycerol, mention may be made of mixtures based on glyceryl stearate, such as the mixture of glyceryl stearate and polyethylene glycol 100 OE monostearate, and in particular the one comprising a 50/50 mixture, sold under the name Arlacel 165® by the company Croda, or else the product containing glyceryl stearate (glyceryl mono-distearate) and potassium stearate, sold under the name Tegin® by the company Goldschmidt (CTFA name: glyceryl stearate SE).

A composition according to the instant disclosure comprises at least one surfactant, in particular a nonionic surfactant, and preferably a surfactant chosen from fatty acid esters of glycerol, nonionic triblock poly(ethylene glycol)-block-poly(propylene glycol)-block-poly(ethylene glycol) copolymers, and mixtures thereof.

Among the nonionic triblock poly(ethylene glycol)-block-poly(propylene glycol)-block-poly(ethylene glycol) copolymers, also referred to as poloxamers, mention may in particular be made of those sold under the name Pluronic or Kolliphor by the company BASF or else under the name of Synperonic by the company Croda.

### Anionic Surfactant

The anionic surfactants may be chosen from alkyl ether sulfates, carboxylates, amino acid derivatives, sulfonates, isethionates, taurates, sulfosuccinates, alkyl sulfoacetates, phosphates and alkyl phosphates, polypeptides, metal salts of C₁₀-C₃₀ and notably C₁₆-C₂₅ fatty acids, in particular metal stearates and behenates, alkali metal salts of cetyl phosphate, and mixtures thereof.

As alkali metal salts of cetyl phosphate, mention may notably be made of potassium cetyl phosphate. Use may notably be made of the monopotassium salt of monocetyl phosphate (INCI name: potassium cetyl phosphate) sold under the name “Amphisol K” by the company DSM Nutritional Products.

### Cationic Surfactant

The cationic surfactants may be chosen from alkylimidazolidiniums, such as isostearyl ethylimidonium ethosulfate, ammonium salts such as (C₁₂-30-alkyl)-tri(C₁-4-alkyl)ammonium halides such as N,N,N-trimethyl-1-docosanaminium chloride (or behentrimonium chloride).

### Amphoteric Surfactant

The compositions according to the instant disclosure may also contain one or more amphoteric surfactants, for instance N-acylamino acids such as N-alkyl aminoacetates and disodium cocoamphodiacetate, and amine oxides such as stearamine oxide, or alternatively silicone surfactants, for instance dimethicone copolyol phosphates such as the product sold under the name Pecosil PS 100@ by the company Phoenix Chemical.

### Silicone Surfactant

The composition may also comprise at least one silicone surfactant. By way of example, as nonionic surfactants with an HLB of greater than or equal to 8 at 25° C., used alone or as a mixture, mention may be made of dimethicone copolyol or dimethicone copolyol benzoate, and as nonionic surfactants with an HLB of less than 8 at 25° C., used alone or as a mixture, mention may be made of the cyclomethicone/dimethicone copolyol mixture.

A composition according to the instant disclosure comprises less than 2% by weight of silicone surfactant(s), in particular less than 1% by weight of silicone surfactant(s), preferably less than 0.5% by weight of silicone surfactant(s) and more preferentially is devoid of silicone surfactant(s).

The surfactant(s) may be present in a composition according to the instant disclosure in a proportion ranging from about 0.05 wt. % to about 15 wt. %, about 0.05 wt. % to about 14 wt. %, about 0.05 wt. % to about 13 wt. %, about 0.05 wt. % to about 12 wt. %, about 0.05 wt. % to about 11 wt. %, about 0.05 wt. % to about 10 wt. %, about 0.1 wt. % to about 15 wt. %, about 0.1 wt. % to 14 wt. %, about 0.1 wt. % to about 13 wt. %, about 0.1 wt. % to about 12 wt. %, about 0.1 wt. % to about 11 wt. %, about 0.1 wt. % to about 10 wt. %, about 0.2 wt. % to about 15 wt. %, about 0.2 wt. % to about 14 wt. %, about 0.2 wt. % to about 13 wt. %, about 0.2 wt. % to about 12 wt. %, about 0.2 wt. % to about 11 wt. %, about 0.2 wt. % to about 10 wt. %, about 0.5 wt. % to about 15 wt. %, about 0.5 wt. % to about 14 wt. %, about 0.5 wt. % to about 13 wt. %, about 0.5 wt. % to about 12 wt. %, about 0.5 wt. % to about 11 wt. %, about 0.5 wt. % to about 10 wt. %, about 1.0 wt. % to about 15 wt. %, about 1.0 wt. % to about 14 wt. %, about 1.0 wt. % to about 13 wt. %, about 1.0 wt. % to about 12 wt. %, about 1.0 wt. % to about 11 wt. %, about 1.0 wt. % to about 10 wt. %, about 1.5 wt. % to about 15 wt. %, about 1.5 wt. % to about 14 wt. %, about 1.5 wt. % to about 13 wt. %, about 1.5 wt. % to about 12 wt. %, about 1.5 wt. % to about 11 wt. %, about 1.5 wt. % to about 10 wt. %, about 2.0 wt. % to about 15 wt. %, about 2.0 wt. % to about 14 wt. %, about 2.0 wt. % to about 13 wt. %, about 2.0 wt. % to about 12 wt. %, about 2.0 wt. % to about 11 wt. %, about 2.0 wt. % to about 10 wt. %, about 2.5 wt. % to about 15 wt. %, about 2.5 wt. % to about 14 wt. %, about 2.5 wt. % to about 13 wt. %, about 2.5 wt. % to about 12 wt. %, about 2.5 wt. % to about 11 wt. %, about 2.5 wt. % to about 10 wt. %, about 2.5 wt. % to about 5 wt. %, including all ranges and sub-ranges there between, based on the total weight of the composition.

### Skin Active Agents

The cosmetic compositions may, optionally, include one or more skin active agents, such as anti-aging agent, anti-wrinkle actives, antioxidants, humectants, moisturizing ingredients, depigmenting agents, and/or agents for treating oily skin etc.

The skin active agents may be included in the cosmetic composition in an amount ranging from greater than zero to about to about 10 wt. %, based on the total weight of the composition. For example, the total amount of skin active agents may be from greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; from about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.05 to about 10 wt. %, about 0.05 to about 5 wt. %, about 0.05 to about 2.5 wt. %, about 0.05 to about 1 wt. %, about 0.05 to about 0.5 wt. %; about 0.1 to about 10 wt. %, about 0.1 to about 5 wt. %, about 0.1 to about 2.5 wt. %, about 0.1 to about 1 wt. %, about 0.1 to about 0.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %, including ranges and subranges therebetween, based on the total weight of the cosmetic composition.

A non-limiting discussion of skin active agents that may, in some cases, be included in the cosmetic composition is provided below:

Humectants and/or Moisturizing Ingredients

Examples of humectants and/or moisturizing ingredients include glycerol and its derivatives, urea and its derivatives, especially Hydrovance marketed by National

Starch, lactic acid, hyaluronic acid, AHA, BHA, sodium pidolate, xylitol, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NutraLipids, a C-glycoside derivatives, in particular the C-13-D-xylopyranoside-2-hydroxypropane in the form of a solution at 30% by weight of active material in a water/propylene glycol mixture (60/40 wt %) as the product produced by the company Chimex under the trade name “Mexoryl SBB”, a rose hip oil marketed by Nestle, a micro-algae extract Prophyridium cruentum enriched with zinc, marketed under the name by Vincience Algualane Zinc spheres of collagen and chondroitin sulfate of marine origin (Atelocollagen) sold by the company Engelhard Lyon under the name Marine Filling Spheres, hyaluronic acid spheres such as those marketed by Engelhard Lyon, and arginine.

### Depigmenting Agents

Depigmenting agents that may be incorporated in the cosmetic composition include those chosen from alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, certain compounds derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

### Anti-Wrinkle Active

The cosmetic composition may include one or more anti-wrinkle actives. The term “anti-wrinkle active” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Exemplary anti-wrinkle actives may be chosen from: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof. Examples of such compounds are: adenosine and its derivatives and retinoids (such as, retinol palmitate and retinol), ascorbic acid and its derivatives such as magnesium ascorbyl phosphate and ascorbyl glucoside; nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-} acetic acid and lipopeptides marketed by the company under the trade names SEDERMA Matrixyl 500 and Matrixyl 3000; lycopene, manganese salts and magnesium salts, especially gluconates, and mixtures thereof. In at least one case, the cosmetic composition includes adenosine derivatives, such as non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside. Other derivatives include adenosine receptor agonists such as adenosine phenylisopropyl (“PIA”), 1-methylisoguanosine, N6-cyclohexyladenosine

(CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′--N-ethylcarboxamido adenosine (CGS-21680), N-ethylcarboxamido-adenosine (NECA), the 5′ (N-cyclopropyl)-carboxamidoadenosine, DPMA (PD 129.944) and metrifudil.

### Skin Active Agent for Oily Skin

The cosmetic composition may, optionally, include a skin active agent that addresses oily skin. These agents can be sebo-regulating or antiseborrhoeic agents capable of regulating the activity of sebaceous glands. Exemplary skin active agents for addressing oily skin include: retinoic acid, retinol, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate;—derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech;—extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C₁₂-C₁₃) sold under the name COSMACOL.RTM. ECI by Sasol; trialkyl citrate (C₁₄-C₁₅) sold under the name COSMACOL.RTM. ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

### Antioxidants

Vitamin C and derivatives may be used, including ascorbic acid, sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. Additionally, extracts from plants containing a high amount of vitamin C such as camu berry (Myrciaria dubia), acerola, emblica officinalis, and bioflavonoids from rose hip and citrus may be used including watersoluble bioflavonoids such as hesperidin methyl chalcone may also be used.

Sesame (Sesamum indicum) or sesame lignan may also be added. Sesame and its lignans (the fibrous compounds associated with the sesame) act as antioxidants. Sesame seed lignans significantly enhance vitamin E activity.

Other antioxidants include tocopherols (e.g. d-α-tocopherol, d-β-tocopherol, d-γ-tocopherol, d-delta-tocopherol), tocotrienols (e.g. d-α-tocotrienol, d-β-tocotrienol, d-γ.-tocotrienol, d-delta-tocotrienol,) and vitamin E (α-tocopherol acetate). These compounds may be isolated from natural sources, prepared by synthetic means, or mixtures thereof. Tocotrienol-enriched vitamin E preparations may be obtained by fractionating vitamin E preparations to remove a portion of tocopherols and recover a preparation more highly concentrated in tocotrienol. Useful tocotrienols are natural products isolated, for example, from wheat germ oil, grain, or palm oil using high performance liquid chromatography, or isolated by alcohol extraction and/or molecular distillation from barley, brewer's grain or oats. As used herein, the term “tocotrienols” includes tocotrienol-rich-fractions obtained from these natural products as well as the pure compounds. The increased glutathione peroxidase activity protects the skin from oxidative damage.

In addition, carotenoids, particularly the xanthophyll type, are also useful antioxidants that can be used. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids.

### Flavonoids

The active agent may be an antioxidant selected from the group of flavonoids. In some instances, the flavonoid is a flavanone (derivative of 2,3-dihydro-2-phenylchromen-4-one). Flavones include: Butin, Eriodictyol, Hesperetin, Hesperidin, Homoeriodictyol, Isosakuranetin, Naringenin, Naringin, Pinocembrin, Poncirin, Sakuranetin, Sakuranin, and Sterubin. The flavonoid may be a flavanonol (derivative of 3-hydroxy-2,3-dihydro-2-phenylchromen-4-one). Flavanols include: Taxifolin, Aromadedrin, Chrysandroside A, Chrysandroside B, Xeractinol, Astilbin, and Fustin. The flavonoid may be a flavone (derivative of 2-phenylchromen-4-one). Flavones include: Apigenin, Luteolin, Tangeritin, Chrysin, Baicalein, Scutellarein, Wogonin, Synthetic Flavones: Diosmin, and Flavoxate. The flavonoid may be a flavonol (derivative of 3-hydroxy-2-phenylchromen-4-one). Flavonols include: 3-Hydroxyflavone, Azaleatin, Fisetin, Galangin, Gossypetin, Kaempferide, Kaempferol, Isorhamnetin,

Morin, Myricetin, Natsudaidain, Pachypodol, Quercetin, Rhamnazin, Rhamnetin, Azalein, Hyperoside, Isoquercitin, Kaempferitrin, Myricitrin, Quercitrin, Robinin, Rutin, Spiraeoside, Xanthorhamnin, Amurensin, Icariin, and Troxerutin. The flavonoid may be a flavan-3-ol (derivatives of 2-phenyl-3,4-dihydro-2H-chromen-3-ol). Flavan-3-ols include: Catechin, Epicatechin, Epigallocatechin, Epicatechin gallate, Epigallocatechin gallate, Epiafzelechin, Fisetinidol, Guibourtinidol, Mesquitol, and Robinetinidol. The flavonoid may be a flavan-4-ol (derivative of 2-phenylchroman-4-ol). Flavan-4-ols include: Apiforol and Luteoforol. The flavonoid may be an isoflavone (derivative of 3-phenylchromen-4-one). Isoflavones include: Genistein, Daidzein, Biochanin A, Formononetin, and the Equol metabolite from Daidzein.

The antioxidant may be an anthocyanidin (derivative of 2-phenylchromenylium cation). Anthocyanidins include: Aurantinidin, Cyanidin, Delphinidin, Europinidin, Luteolinidin, Pelargonidin, Malvidin, Peonidin, Petunidin, Rosinidin, and Xanthone.

The antioxidant may be a Dihydrochalcone (derivative of 1,3-diphenyl-1-propanone). Dihydrochalcones include: Phloretin, Dihydrochalcone phloretin Phlorizin, Aspalathin, Naringin dihydrochalcone, Neohesperidin dihydrochalcone, and Nothofagin. Without limiting the mode of action of the invention, dihydrochalcones may exert an antioxidant effect by reducing reactive free radicals, like reactive oxygen and reactive nitrogen species.

The antioxidant may be an anthocyanin. Anthocyanins and their derivatives are antioxidants. Anthocyanins encompasses a class of flavonoid compounds that are naturally occurring, water-soluble compounds, responsible for the red, purple, and blue colors of many fruits, vegetables, cereal grains, and flowers. Additionally, anthocyanins are collagenase inhibitors. The inhibition of collagenase helps in the prevention and reduction of wrinkles, increase in skin elasticity, etc., which are caused by a reduction in skin collagen. The anthocyanins may be obtained from any portion of various plant sources, such as the fruit, flower, stem, leaves, root, bark, or seeds. One of skill in the art will understand that certain portions of the plant may contain higher natural levels of anthocyanins, and, therefore, those portions are used to obtain the desired anthocyanins. In some instances, antioxidants may include one or more betacyanin. Betacyanins, like anthocyanins, may be obtained from natural sources and are antioxidants.

The antioxidant may be a Phenylpropanoid (derivatives of cinnamic acid). Phenylpropanoids include: Cinnamic acid, Caffeic acid, Ferulic acid, Trans-ferulic acid (including its antioxidant pharmacore 2,6-dihydroxyacetophenome), 5-Hydroxyferulic acid, Sinapic acid, Coumaryl alcohol, Coniferyl alcohol, Sinapyl alcohol, Eugenol, Chavicol, Safrole, P-coumaric acid, and Sinapinic acid. Without limiting the mode of action of the invention, Phenylpropanoids may neutralize free radicals.

The antioxidant may be a Chalcone (derivative of 1,3-diphenyl-2-propen-1-one). Chalcones include: Butein, Okanin, Carthamin, Marein, Sophoradin, Xanthohumol, Flavokvain A, Flavokavain B, Flavokavin C, and synthetic Safalcone.

The antioxidant may be a Curcuminoid. Curcuminoids include: Curcumin, Desmethoxycurcumin, bis-Desmethoxycurcumin, Tetrahydrocurcumin, and Tetrahydrocurcuminoids. Curcumin and tetrahydrocurcuminoids may be derived from rhizomes of Curcuma longa. Tetrahydrocurcumin, a metabolite of curcumin, has been found to be a more potent antioxidant and more stable compared to curcumin.

The antioxidant may be a Tannin. Tannins include: Tannin, Terflavin B, Glucogallin, Dgallic acid, and Quercitannic acid.

The antioxidant may be a stilbenoid. Stilbenoids include: Resveratrol, Pterostilbene, and Piceatannol. Resveratrol may include, but is not limited to, 3,5,4′-trihydroxystilbene, 3,4,3′,5′-tetrahydroxystilbene (piceatannol), 2,3′,4,5′-tetrahydroxystilbene (oxyresveratrol), 4,4′-dihydroxystilbene, and alpha and beta glucoside, galactoside and mannoside derivatives thereof.

The antioxidant may be a Coumarin (derivatives of 2H-chromen-2-one). Coumarins include: 4-Hydroxycoumarin, Umbelliferone, Aesculetin, Herniarin, Auraptene, and Dicoumarol.

The antioxidant may be a Carotenoid. Carotenoids include: beta-Carotene, alpha-Carotene, gamma-Carotene, beta-Cryptoxanthin, Lycopene, Lutein, and Idebenone. Sesame (Sesamum indicum) or sesame lignan may also be added. Sesame and its lignans (the fibrous compounds associated with the sesame) act as antioxidants. Sesame seed lignans significantly enhance vitamin E activity.

The antioxidant may be: a Xanthone, Butylated Hydroxytoluene, 2,6-Di-tert-butylphenol, 2,4-Dimethyl-6-tert-butylphenol, Gallic acid, Eugenol, Uric acid, alpha-Lipoic acid, Ellagic acid, Chicoric acid, Chlorogenic acid, Rosmarinic acid, Salicylic acid, Acetylcysteine, S-Allyl cysteine, Barbigerone, Chebulagic acid, Edaravone, Ethoxyquin, Glutathione, Hydroxytyrosol, Idebenone, Melatonin, N-Acetylserotonin, Nordihydroguaiaretic acid, Oleocanthal, Oleuropein, Paradol, Piceatannol, Probucol, Propyl gallate, Protocatechuic acid, Pyritinol, Rutin, Secoisolariciresinol diglucoside, Sesamin, Sesamol, Silibinin, Silymarin, Theaflavin, Theaflavin digallate, Thmoquinone, Trolox, Tyrosol, Polyunsaturated fatty acids, and sulfur-based antioxidants such as Methionine or Lipoic acid.

### C-glycoside(s)

The cosmetic compositions may include a C-glycoside, for instance, as a skin active ingredient. For example, a C-glycoside in an amount of about 0.1 to about 10 wt. %, based on the total weight of the cosmetic composition. For example, the amount of C-glycoside present in the cosmetic composition may be from about 0.1 to about 10 wt. %, about 0.1 to about 9 wt. %, about 0.1 to about 8 wt. %, about 0.1 to about 7 wt. %, about 0.1 to about 6 wt. %, about 0.1 to about 5 wt. %, about 0.1 to about 4 wt. %; about 1 to about 10 wt. %, about 1 to about 9 wt. %, about 1 to about 8 wt. %, about 1 to about 7 wt. %, about 1 to about 6 wt. %, about 1 to about 5 wt. %, about 1 to about 4 wt. %;

about 2 to about 10 wt. %, about 2 to about 9 wt. %, about 2 to about 8 wt. %, about 2 to about 7 wt. %, about 2 to about 6 wt. %, about 2 to about 5 wt. %, about 2 to about 4 wt. %; about 3 to about 10 wt. %, about 3 to about 9 wt. %, about 3 to about 8 wt. %, about 3 to about 7 wt. %, about 3 to about 6 wt. %, about 3 to about 5 wt. %, or about 3 to about 4 wt. %, based on the total weight of the cosmetic composition.

The cosmetic composition may include one or more C-glycoside(s) having a structure in accordance with general formula (I):

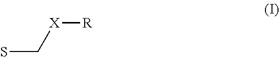

in which:
R represents a saturated C₁ to C₁₀, in particular C₁ to C₄, alkyl radical which can optionally be substituted by at least one radical chosen from OH, COOH or COOR″₂, with R″₂ being a saturated C₁-C₄ alkyl radical,
S represents a monosaccharide or a polysaccharide comprising up to 20 sugar units, in particular up to 6 sugar units, in pyranose and/or furanose form and of the L and/or D series, it being possible for the said monosaccharide or polysaccharide to be substituted by a hydroxyl group which is necessarily free and optionally one or more optionally protected amine functional group(s), and
X represents a radical chosen from the —CO—, —CH(OH)—, —CH(NH₂)—, —CH (NHCH₂CH₂CH₂OH)-, —CH(NHPh)- and —CH(CH₃)- groups and in particular a —CO—, —CH(OH)— or —CH(NH₂)—radical and more particularly a —CH(OH)— radical,
wherein the S-CH2-X bond represents a bond of C-anomeric nature, which can be α or β, and also their physiologically acceptable salts, their solvates, such as the hydrates, and their optical and geometrical isomers.

The C-glycosides of formula (I) preferably have a structure, wherein R denotes a saturated linear C₁ to C₆, in particular C₁ to C₄, preferentially C₁ to C₂, alkyl radical and more preferably a methyl radical. Mention may, in particular, be made of the following alkyl groups for the C-glycosides (such as R): methyl, ethyl, isopropyl, n-propyl, n-butyl, t-butyl, isobutyl, sec-butyl, pentyl, n-hexyl, cyclopropyl, cyclopentyl and cyclohexyl groups.

According to at least one embodiment, use may be made of a C-glycoside compound corresponding to the formula (I) for which S represents a monosaccharide or a polysaccharide comprising up to 6 sugar units, such as in pyranose and/or furanose form and of L and/or D chimeric series. The mono-or polysaccharide exhibits at least one free hydroxyl functional group and/or optionally one or more protected amine functional groups, where X and R otherwise retain all of the definitions given above.

Advantageously, a monosaccharide of the invention can be chosen from D-glucose, D-galactose, D-mannose, D-xylose, D-lyxose or L-fucose, L-arabinose, L-rhamnose, D-glucuronic acid, D-galacturonic acid, D-iduronic acid, N-acetyl-D-glucosamine or N-acetyl-D-galactosamine and advantageously denotes D-glucose, D-xylose, N-acetyl-D-glucosamine, L-fucose, and a mixture thereof. In one instance, the monosaccharide is D-xylose.

More particularly, a polysaccharide of the invention comprising up to 6 sugar units can be chosen from D-maltose, D-lactose, D-cellobiose, D-maltotriose, a disaccharide combining an uronic acid chosen from D-iduronic acid or D-glucuronic acid with a hexosamine chosen from D-galactosamine, D-glucosamine, N-acetyl-D-galactosamine or N-acetyl-D-glucosamine, an oligosaccharide comprising at least one xylose that can advantageously be chosen from xylobiose, methyl-β-xylobioside, xylotriose, xylotetraose, xylopentaose, xylohexaose and a mixture thereof. Preferably, the oligosaccharide is xylobiose, which is composed of two xylose molecules linked via a 1-4 bond. More particularly, S can represent a monosaccharide chosen from D-glucose, D-xylose, L-fucose, D-galactose, D-maltose, and a mixture thereof. In some cases, the monosaccharide is D-xylose.

Preferably, the C-glycoside may be a derivative of formula (I) for which:
R denotes an unsubstituted linear C₁-C₄, in particular C₁-C₂, alkyl radical, especially a methyl radical;
S represents a monosaccharide as described above and chosen in particular from D-glucose, D-xylose, N-acetyl-D-glucosamine or L-fucose, and in particular D-xylose; and
X represents a group chosen from —CO—, —CH(OH)— or —CH(NH₂)- and preferably a —CH(OH)— group.

The compounds described herein may be in the form of a salt. Acceptable salts of the compounds described in the present disclosure comprise conventional non-toxic salts of the said compounds, such as those formed from organic or inorganic acids. Mention may be made, by way of example, of the salts of inorganic acids, such as sulfuric acid or hydrochloric acid. Mention may also be made of the salts of organic acids, which can comprise one or more carboxylic, sulfonic or phosphonic acid groups. Mention may in particular be made of propionic acid, acetic acid, terephthalic acid, citric acid and tartaric acid.

When the compound of formula (I) comprises an acid group, neutralization of the acid group(s) can be carried out with an inorganic base, such as LiOH, NaOH, KOH, Ca(OH)₂, NH₄OH, Mg(OH)₂ or Zn(OH)₂, or with an organic base, such as a primary, secondary or tertiary alkylamine. For example, the compound of formula (I) may be neutralized with triethylamine or butylamine. This primary, secondary or tertiary alkylamine can comprise one or more nitrogen and/or oxygen atoms and can comprise, e.g., one or more alcohol functional groups. Mention may be made of 2-amino-2-methylpropanol, triethanolamine, 2-(dimethylamino) propanol or 2-amino-2-(hydroxymethyl)-1,3-propanediol. Mention may also be made of lysine or 3-(dimethylamino)propylamine.

Non-limiting examples of solvates which may be used in the cosmetic composition include conventional solvates, such as those formed during the final stage of preparation of said compounds due to the presence of solvents. Mention may be made, by way of example, of the solvates due to the presence of water or of linear or branched alcohols, such as ethanol or isopropanol. Of course, according to the disclsoure, a C-glycoside derivative corresponding to the formula (I) can be used alone or as a mixture with other C-glycoside derivatives and in any proportion.

A C-glycoside derivative which is suitable for the invention can in particular be obtained by the synthetic method described in the document WO 02/051828, which is incorporated herein in its entirety for all purposes. Non-limiting examples of C-glycoside compounds which may be suitable for the cosmetic composition include:
C-β-D-xylopyranoside-n-propane-2-one,
C-α-D-xylopyranoside-n-propan-2-one,
C-β-D-xylopyranoside-2-hydroxypropane,
C-α-D-xylopyranoside-2-hydroxypropane,
1-(C-β-D-fucopyranoside) propan-2-one,
1-(C-α-D-fucopyranoside) propan-2-one,
1-(C-B-L-fucopyranoside) propan-2-one,
1-(C-α-L-fucopyranoside) propan-2-one,
1-(C-β-D-fucopyranoside)-2-hydroxypropane,
1-(C-α-D-fucopyranoside)-2-hydroxypropane,
1-(C-B-L-fucopyranoside)-2-hydroxypropane,
1-(C-α-L-fucopyranoside)-2-hydroxypropane,
1-(C-β-D-glucopyranosyl)-2-hydroxypropane,
1-(C-α-D-glucopyranosyl)-2-hydroxypropane,
1-(C-β-D-galactopyranosyl)-2-hydroxypropane,
1-(C-α-D-galactopyranosyl)-2-hydroxypropane,
1-(C-β-D-fucofuranosyl) propan-2-one,
1-(C-α-D-fucofuranosyl) propan-2-one,
1-(C-B-L-fucofuranosyl) propan-2-one,
1-(C-α-L-fucofuranosyl) propan-2-one,
C-β-D-maltopyranoside-n-propane-2-one,
C-α-D-maltopyranoside-n-propan-2-one,
C-β-D-maltopyranoside-2-hydroxypropane,
C-α-D-maltopyranoside-2-hydroxypropane, isomers therof, and/or mixtures thereof.

The cosmetic composition may, preferably, include a C-glycoside chosen from C-β-D-xylopyranoside-2-hydroxypropane, C-α-D-xylopyranoside-2-hydroxypropane, and a mixture thereof. In some cases, the C-glycoside is C-β-D-xylopyranoside-2-hydroxypropane. According to at least one embodiment, the C-glycoside compound can be C-β-D-xylopyranoside-2-hydroxypropane (or hydroxypropyl tetrahydropyrantriol), which may be provided in the form of a solution containing 30% by weight of active.

### Water-Soluble Solvents

In various embodiments, the compositions may include one or more water soluble solvents. In some embodiments, the one or more water soluble solvents are selected form the group consisting of glycerin, C₂-C₅ mono-alcohols, polyols (polyhydric alcohols), glycols, and a mixture thereof.

The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90%. Non-limiting examples of water-soluble solvents include, for example, glycerin, alcohols (for example, C₁₋₃₀, C₁₋₁₅, C₁₋₁₀, or C₁₋₄ alcohols), organic solvents, polyols (polyhydric alcohols), glycols (e.g., butylene glycol, caprylyl glycol, etc.), and a mixture thereof.

In some cases, the water-soluble solvent is a monoalcohol. Non-limiting examples of monoalcohols include ethanol, propanol, butanol, pentanol, hexanol, isopropyl alcohol, cyclohexanol, isobutyl alcohol, 2-methyl-2-butanol (2-methylbutan-2-ol), and a mixture thereof. In some instances, the monoalcohols comprise or are chosen from ethanol, propanol, butanol, pentanol, an isomer thereof, or a combination thereof. In further instances, the one or more monoalcohol(s) includes or consists of ethanol.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. The water-soluble solvents may be organic solvents that can be volatile or non-volatile compounds.

Further non-limiting examples of water-soluble solvents include alkanediolssuch as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. The term “polyol” should be understood as meaning, within the meaning of the present disclosure, an organic molecule comprising at least two free hydroxyl groups. The polyols of the cosmetic composition may be glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Non-limiting examples of polyols that may, optionally, be included in the cosmetic composition include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof. The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol.

In some cases, the polyol comprises glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

The total amount water soluble solvents may vary but is typically from about 0.1 to about 30 wt. %, based on the total weight of the composition. For example, the total amount of water soluble solvents may be from about 0.1 to about 25 wt. %, about 0.1 to about 20 wt. %, from about 0.1 to about 15 wt. %, about 0.1 to about 10 wt. %, about 0.1 to about 8 wt. %, about 0.1 to about 6 wt. %, from about 0.5 to about 25 wt. % about 0.5 to about 20 wt. %, about 0.5 to about 15 wt. %, about 0.5 to about 10 wt. %, about 0.5 to about 8 wt. %, about 0.5 to 6 wt. %, from about 1 to about 25 wt. %, about 1 to about 20 wt. %, about 1 to about 15 wt. %, about 1 to about 10 wt. %, about 1 to about 8 wt. %, or about 1 to about 6 wt. %, from about 1.5 to about 25 wt. %, about 1.5 to about 20 wt. %, about 1.5 to about 15 wt. %, about 1.5 to about 10 wt. %, about 1.5 to about 8 wt. %, or about 1.5 to about 6 wt. %, from about 2 to about 25 wt. %, about 2 to about 20 wt. %, about 2 to about 15 wt. %, about 2 to about 10 wt. %, about 2 to about 8 wt. %, or about 2 to about 6 wt. %, from about 2.5 to about 25 wt. %, about 2.5 to about 20 wt. %, about 2.5 to about 15 wt. %, about 2.5 to about 10 wt. %, about 2.5 to about 8 wt. %, or about 2.5 to about 6 wt. %, from about 3 to about 25 wt. %, about 3 to about 20 wt. %, about 3 to about 15 wt. %, about 3 to about 10 wt. %, about 3 to about 8 wt. %, or about 3 to about 6 wt. %, from about 3.5 to about 25 wt. %, about 3.5 to about 20 wt. %, about 3.5 to about 15 wt. %, about 3.5 to about 10 wt. %, about 3.5 to about 8 wt. %, or about 3.5 to about 6 wt. %, including ranges and sub-ranges therebetween, based on the total weight of the composition. Additionally or alternatively, the total amount of water soluble solvents may be from 0.1 to 25 wt. %, 0.1 to 20 wt. %, from 0.1 to 15 wt. %, 0.1 to 10 wt. %, 0.1 to 8 wt. %, 0.1 to 6 wt. %, from 0.1 to 25 wt. %, 0.5 to 20 wt. %, 0.5 to 15 wt. %, 0.5 to 10 wt. %, 0.5 to 8 wt. %, 0.5 to 6 wt. %, from 1 to 25 wt. %, 1 to 20 wt. %, 1 to 15 wt. %, 1 to 10 wt. %, 1 to 8 wt. %, or 1 to 6 wt. %, from 1.5 to 25 wt. %, 1.5 to 20 wt. %, 1.5 to 15 wt. %, 1.5 to 10 wt. %, 1.5 to 8 wt. %, or 1.5 to 6 wt. %, from 2 to 25 wt. %, 2 to 20 wt. %, 2 to 15 wt. %, 2 to 10 wt. %, 2 to 8 wt. %, or 2 to 6 wt. %, from 2.5 to 25 wt. %, 2.5 to 20 wt. %, 2.5 to 15 wt. %, 2.5 to 10 wt. %, 2.5 to 8 wt. %, or 2.5 to 6 wt. %, from 3 to 25 wt. %, 3 to 20 wt. %, 3 to 15 wt. %, 3 to 10 wt. %, 3 to 8 wt. %, or 3 to 6 wt. %, from 3.5 to 25 wt. %, 3.5 to 20 wt. %, 3.5 to 15 wt. %, 3.5 to 10 wt. %, 3.5 to 8 wt. %, or 3.5 to 6 wt. %, including ranges and sub-ranges therebetween, based on the total weight of the composition.

The instant disclosure also relates to methods of using the compositions described herein. For example, the compositions can be used in a method that comprises applying the cosmetic compositions to the skin of humans. In some cases, the composition is applied to the face.

It is to be understood that both the foregoing general description and the following detailed description are exemplary and explanatory only, and are not restrictive of the invention.

### EXAMPLES

As various changes could be made in the above-described methods without departing from the scope of the instant disclosure, it is intended that all matter contained in the above description and in the examples given below, shall be interpreted as illustrative and not in a limiting sense. The ingredient amounts in the compositions/formulas described below are expressed in % by weight, based on the total weight of the composition.

### Example 1

As various changes could be made in the above-described methods without departing from the scope of the instant disclosure, it is intended that all matter contained in the above description and in the examples given below, shall be interpreted as illustrative and not in a limiting sense.

The following Examples are intended to be non-restrictive and explanatory only. The ingredient amounts in the compositions/formulas described below are expressed in % by weight, based on the total weight of the composition.

The base formula, the inventive compositions and the comparative compositions were prepared as described below.

### Preparation of the Compositions

The water and water soluble solvents were stirred in a vessel of appropriate size and heated to 75C. The fatty compounds, surfactants, emollients, and any oil soluble actives were combined and heated in a side vessel until 75° C. and all components completely melted. The fatty phase was then added to the water phase at 75° C. with stirring and homogenization for 15 minutes. After this time, the vessel was cooled to room temperature, with sitting and homogenization constant. Below 40° C., polymeric thickeners and fillers were added. Stirring and homogenization continued until polymers were well dispersed. Then, pH was adjusted and remaining miscellaneous raw materials were added. Once homogenous, batch was completed and removed from kettle at 20° C.

TABLE 1

| | | | Base | Inventive | Comp. | Comp. | Comp. |
|---|---|---|---|---|---|---|---|
| | | INCI US | Formula | Example | Ex. 1 | Ex. 2 | Ex. 3 |
| a | Emollient | LAURYL LAURATE | 0 | 0.70 | 0 | 1.00 | 0 |
| | | BUTYROSPERMUM PARKII (SHEA) | 0 | 0 | 0 | 0 | 0.7 |
| | | BUTTER | | | | | |
| b | Oil Phase | POLY C10-30 ALKYL ACRYLATE¹ | 0 | 0.30 | 1.00 | 0 | 0.30 |
| | Thickener | | | | | | |
| | Polymeric | CARBOMER | 0.15 | 0.15 | 0.15 | 0.15 | 0.15 |
| | Thickener | ACRYLATES/C10-30 ALKYL | 0.15 | 0.15 | 0.15 | 0.15 | 0.15 |
| | | ACRYLATE CROSSPOLYMER | | | | | |
| | Surfactant | ARACHIDYL GLUCOSIDE | 0.08 | 0.08 | 0.08 | 0.08 | 0.08 |
| | | BEHENYL ALCOHOL | 0.15 | 0.15 | 0.15 | 0.15 | 0.15 |
| | | ARACHIDYL ALCOHOL | 0.28 | 0.28 | 0.28 | 0.28 | 0.28 |
| | | GLYCERYL STEARATE | 0.25 | 0.25 | 0.25 | 0.25 | 0.25 |
| | | PEG-100 STEARATE | 0.25 | 0.25 | 0.25 | 0.25 | 0.25 |
| | Fatty | SQUALANE; ISOPROPYL LAUROYL | 4.00 | 4.00 | 4.00 | 4.00 | 4.00 |
| | Compound | SARCOSINATE; PENTAERYTHRITYL | | | | | |
| | | TETRAISOSTEARATE; CAPRYLIC/ | | | | | |
| | | CAPRIC TRIGLYCERIDE | | | | | |
| | Filler | CELLULOSE | 0.3 | 0.3 | 0.3 | 0.3 | 0.3 |
| | Active | SODIUM HYALURONATE | 0.10 | 0.10 | 0.10 | 0.10 | 0.10 |
| | Compound | NIACINAMIDE | 5.00 | 5.00 | 5.00 | 5.00 | 5.00 |
| | | HYDROXYACETOPHENONE | 0.30 | 0.30 | 0.30 | 0.30 | 0.30 |
| | Water- | BUTYLENE GLYCOL | 3.00 | 3.00 | 3.00 | 3.00 | 3.00 |
| | Soluble | PENTYLENE GLYCOL | 4.00 | 4.00 | 4.00 | 4.00 | 4.00 |
| | Solvent | GLYCERIN | 8.00 | 8.00 | 8.00 | 8.00 | 8.00 |
| | Solvent | WATER | 73.32 | 72.32 | 72.32 | 72.32 | 72.32 |

| | | | | | |
|---|---|---|---|---|---|
| Additional Miscellaneous Ingredients such as | less | less | less | less | less |
| preservatives, fragrances, pH adjusters, chelants, | than 10 | than 10 | than 10 | than 10 | than 10 |
| etc.² | | | | | |

¹Intelimer ® IPA13-1/Evonik
²For example, phenoxyethanol (preservatives); sodium hydroxide (pH adjuster); trisodium ethylenediamine disuccinate (chelant).

### Example 2

### (Tackiness)

### Protocol for the Sensory Study

50 uL of a blinded test sample was deposited on the back of the test subject's hand. Using two fingers in a controlled motion, the sample was spread in a circular manner, with the test subject completing the circular motion 20 times. The sensory of the test sample was evaluated on back of hand 1) during application, 2) immediately after application, and 3) two minutes after application. Test subjects were asked to score three overall attributes on a scale from 1-5.

The compositions presented in Example 1 were tested. The criteria tested and assessments are reported in Table 2 below. The base formula was a gel cream formulated and known to exhibit tackiness (Table 1, base formula). The tackiness mostly came from the 8% of glycerin and 5% of niacinamide.

The results are presented in the table below.

TABLE 2
Sensory Results

| | Trial | Concentration | Playtime | Greasiness | Tack | Sensory |
|---|---|---|---|---|---|---|
| Base Formula | Base Fla | N/A | 4 | 2 | 4 | tacky |
| Inv. Ex. | Poly C10-30 | 0.3%, 0.7% | 4 | 1 | 1 | soft |
| | Alkyl Acrylate, | | | | | |
| | Lauryl laurate | | | | | |
| Comp. Ex. 1 | Poly C10-30 | 1% | 4 | 2 | 2 | waxy, |
| | Alkyl Acrylate | | | | | filmy |
| Comp. Ex. 2 | Lauryl laurate | 1% | 4 | 2.5 | 3.5 | dry |
| | alone | | | | | |
| Comp. Ex. 3 | Poly C10-30 | 0.3%, 0.7% | 4 | 4 | 2 | greasy |
| | Alkyl Acrylate, | | | | | |
| | Shea butter | | | | | |
| | | | 1 = volatile, | 1 = no | 1 = no tack, | |
| | | | fast absorbing, | oiliness | clean | |
| | | | short rubbing | | | |
| | | | 5 = persistent, | 5 = oily/ | 5 = high | |
| | | | gliding, long | greasy | tack, sticky | |
| | | | rubbing | | | |

The sensory results demonstrated that the combination of lauryl laurate and Poly C10-30 Alkyl Acrylate in a gel cream base exhibited the least amount of tackiness with the best sensory attributes. When the formula contained only the Poly C10-30 Alkyl

Acrylate (i.e. Comp. Ex.1), the formula exhibited an overall feel of waxy, filmy sensory and was somewhat tacky. When the formula contained only the lauryl laurate (i.e. Comp. Ex.2), the formula exhibited an overall end feel of dryness and was tacky and sticky. When the formula contained the Poly C10-30 Alkyl Acrylate associated with an emollient comparable to the lauryl laurate (i.e. Comp. Ex. 3; shea butter melts above room temperature, but close to skin temperature (peak melting at 26° C., melting endset at 34° C.). If melting temperatures dictated/predicted the sensory characteristics, then the formulas comparing shea butter and lauryl laurate would be very similar, but that is not the case), the formula exhibited an overall end feel of greasiness and was somewhat tacky.

The foregoing description illustrates and describes the instant disclosure. The disclosure shows and describes only the preferred embodiments but it should be understood that the invention is capable to use in various other combinations, modifications, and environments and is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein.

### DEFINITIONS

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the claimed invention.

The terms “a,” “an,” and “the” are understood to encompass the plural as well as the singular.

The term “a mixture thereof” (or “combination thereof”) is interchangeable with the term “mixtures thereof” (or “combinations thereof”). Throughout the disclosure, if the term “a mixture thereof” is used, following a list of elements as shown in the following example where letters A-F represent the elements: “one or more elements selected from A, B, C, D, E, F, or mixtures thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

An “alkyl radical” is a linear or branched saturated hydrocarbon-based group, particularly C1-C8, more particularly C1-C6, preferably C1-C4 such as methyl, ethyl, isopropyl and tert-butyl.

An “alkoxy radical” is a alkyl-oxy wherein alkyl is as described herein before.

An “alkenyl radical” is a linear or branched unsaturated hydrocarbon-based group, particularly C2-C8, more particularly C2-C6, preferably C2-C4 such as ethylenyl, propylenyl.

An “alkylene radical” is a linear or branched divalent saturated C1-C8, in particular C1-C6, preferably C1-C4 hydrocarbon-based group such as methylene, ethylene or propylene.

Some of the various categories of components identified for the cosmetic compositions may overlap. In such cases where overlap may exist and the composition/product includes two overlapping components (or more than two overlapping components), an overlapping component does not represent more than one component. As an example, tocopherol may be considered both a “vitamin” and a “preservative.” If a particular composition/product includes both a vitamin and a preservative, tocopherol can serve as only a vitamin or only a preservative (tocopherol does not simultaneously serve as both the vitamin and the preservative, even though it functionally provides more than one effect).

All percentages, parts and ratios herein are based upon the total weight of the compositions of the present invention, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically points 1, 2, 3, 4 and 5, as well as sub-ranges such as 2-5, 3-5, 2-3, 2-4, 1-4, etc.; and points of 1, 2, 3, 4, and 5 includes ranges and sub-ranges of 1-5, 2-5, 3-5, 2-3, 2-4, 1-4, etc.

Other than in the operating examples, or where otherwise indicated, all numbers expressing quantities of ingredients and/or reaction conditions may be modified with the term “about,” whether or not expressly stated.

Additionally, all numbers are intended to represent exact values as additional embodiments, whether or not modified by the term “about.” For example, “an amount of about 1%” can be modified to refer to exactly 1%. As a further example, “an amount of 1%” can be modified to refer to “about 1%.” Unless otherwise indicated, the term “about” is understood to encompass a range of +/−5% from the stated number. However, in some embodiments, the term may be defined to encompass narrower ranges, for example, +/−1%, 2%, 3%, or 4% from the stated number.

Various components may be included as salts even if not explicitly stated. In other words, whenever a compound has the ability to be in the form of a salt, it is intended that salts are also encompassed to the extent such salts exist, even though the specification may not specifically refer to a salt (or may not refer to a salt in every instance throughout the disclosure), for example, by using language such as “a salt thereof” or “salts thereof.” Sodium and potassium are common cations that form salts. However, additional cations such as ammonium ions, or alkanolammonium ions such as monoethanolammonium or triethanolammonium ions, may also form salts.

The term “substantially free” or “essentially free” as used herein means the specific material may be present in small amounts that do not materially affect the basic and novel characteristics of the claimed invention. For instance, there may be less than 2% by weight of a specific material added to a composition, based on the total weight of the composition (provided that an amount of less than 2% by weight does not materially affect the basic and novel characteristics of the claimed invention). Similarly, a composition “substantially free” or “essentially free” of a stated material may include less than 1.5 wt. %, less than 1 wt. %, less than 0.5 wt. %, less than 0.1 wt. %, less than 0.05 wt. %, or less than 0.01 wt. %, or none of the specified material. The term “substantially free” or “essentially free” as used herein may also mean that the specific material is not added to the composition but may still be present in a raw material that is included in the composition.

Furthermore, all components that are positively set forth in the instant disclosure may be negatively excluded from the claims, e.g., a claimed composition may be “free,” “essentially free” (or “substantially free”) of one or more components that are positively set forth in the instant disclosure. As an example, silicones can optionally be included in the cosmetic compositions but in some instances the compositions may be free or essentially free from silicones. Silicones are synthetic polymers made up of repeating units of siloxane, elemental silicon and oxygen, combined with other elements, most often carbon and hydrogen. Thus, silicones are also called polysiloxanes. In some instances, cosmetic compositions of the instant case can be free or essentially free from dimethicones, amomdimethicones, dimethiconols, cyclosiloxanes, siloxanes, etc. All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) lauryl laurate;
(b) at least one semicrystalline polymer containing at least one alkyl acrylate chain.

2. The composition of claim 1, wherein lauryl laurate is present from about 0.1 to about 5 wt. %, based on a total weight of the composition.

3. The composition of claim 1, wherein the at least one semicrystalline polymer(s) containing at least one alkyl acrylate chain are chosen from the homopolymers obtained by polymerization of a monomer chosen from alkyl acrylates and alkyl methacrylates and from the copolymers obtained by copolymerization of a monomer chosen from alkyl acrylates and alkyl methacrylates with a hydrophilic monomer, preferably a hydroxyethyl (meth)acrylate, and mixtures thereof.

4. The composition of claim 1, wherein the semicrystalline polymer(s) containing at least one alkyl acrylate chain are chosen from a polystearyl acrylate, a polybehenyl acrylate, a behenyl acrylate/2-hydroxyethyl acrylate copolymer, a stearyl acrylate/2-hydroxyethyl acrylate copolymer, and one of the mixtures thereof.

5. The composition of claim 4, wherein the semicrystalline polymer(s) containing at least one alkyl acrylate chain at least one of the one or more intelimer is selected from the group consisting of Poly C10-30 Alkyl Acrylate, C12-22 Alkyl Acrylate/Hydroxyethylacrylate Copolymer, and mixtures thereof.

6. The composition of claim 1, wherein the content of semicrystalline polymer(s) containing at least one alkyl acrylate chain is present from about 0.1 to about 3 wt. % based on a total weight of the composition.

7. The composition of claim 1, further comprising one or more polymeric thickener.

8. The composition of claim 7, wherein the one or more polymeric thickeners are present from about 0.05 to about 5 wt. % based on a total weight of the composition.

9. The composition of claim 1, further comprising one or more fatty compounds.

10. The composition of claim 1, wherein the one or more fatty compounds are present from about 0.1 to about 30 wt. % based on a total weight of the composition.

11. The composition of claim 1, further comprising one or more surfactants.

12. The composition of claim 10, wherein the one or more surfactants are present from about 0.05 to about 15 wt. % based on a total weight of the composition.

13. The composition of claim 1, further comprising one or more skin active agents.

14. The composition of claim 13, wherein the total amount of the skin active agents is from at least about 1 wt. % based on a total weight of the composition.

15. The composition of claim 1, further comprising one or more water soluble solvents.

16. The composition of claim 1, wherein the one or more water soluble solvents are present from about 0.1 to about 30 wt. % based on a total weight of the composition.

17. The composition of claim 15, wherein one or more water soluble solvents are selected form the group consisting of glycerin, C2-C5 mono-alcohols, polyols (polyhydric alcohols), glycols, and a mixture thereof.

18. A cosmetic composition comprising:
(a) about 0.1 to about 5 wt. % of lauryl laurate;
(b) about 0.1 to about 3 wt. % poly C10-30 alkyl acrylate;
(c) about 0.05 to about 5 wt. % of one or more polymeric thickeners;
(d) about 0.05 to about 15 wt. % of one or more surfactants;
(e) about 0.05 to about 15 wt. % of one or more fatty compounds;
(f) about 0.1 to about 20 wt. % of one or more skin active agents;
(g) about 0.1 to about 30 wt. % of one or more water soluble solvents selected from glycerin, C₂-C₅ mono-alcohols, polyols (polyhydric alcohols), glycols, and a mixture thereof;
wherein all weight percentages are based on a total weight of the composition.

19. A method for treating skin comprising apply the composition of claim 1 to the skin.
